(19) United States
(12) Patent Application Publication
(10) Pub. No.: US 2024/0424496 A1
(43) Pub. Date: Dec. 26, 2024

(54) Digital Microfluidic Device, Drive Method and Use Thereof

(71) Applicants: Beijing BOE Sensor Technology Co., Ltd., Beijing (CN); BOE Technology Group Co., Ltd., Beijing (CN)

(72) Inventors: Yingying ZHAO, Beijing (CN); Le GU, Beijing (CN); Bolin FAN, Beijing (CN); Yongjia GAO, Beijing (CN); Huazhe LIU, Beijing (CN); Li YANG, Beijing (CN)

(21) Appl. No.: 18/699,281

(22) PCT Filed: Sep. 25, 2023
(86) PCT No.: PCT/CN2023/121260

§ 371 Date: Apr. 7, 2024

(30) Foreign Application Priority Data
Sep. 23, 2022 (WO) PCT/CN2022/120866

(51) Int. Cl. B01L 3/00

(52) U.S. Cl. CPC B01L 3/502792; B01L 2200/0652; B01L 2300/0654; B01L 2300/161; B01L 2300/18; B01L 2400/0415

(57) ABSTRACT

Provided in the present disclosure are a digital micro-fluidic apparatus, a driving method therefor, and the use thereof. The digital micro-fluidic apparatus comprises a digital micro-fluidic chip, the digital micro-fluidic chip at least comprising a drive electrode and a reference electrode, and the reference electrode being configured to write in a first reference voltage. The drive electrode is configured to alternately write in a first scanning voltage and a second scanning voltage so as to be alternately in an actuated state and a non-actuated state. In the actuated state, the drive electrode is configured to actuate composite liquid drops present therein; and in the non-actuated state, the drive electrode is configured to not actuate the composite liquid drops present therein.

### CROSS-REFERENCE TO RELATED APPLICATIONS

The present disclosure is a U.S. National Phase Entry of International Application PCT/CN2023/121260 filed on Sep. 25, 2023, which claims priority to PCT International Patent Application No. PCT/CN2022/120866, filed to the CNIPA on Sep. 23, 2022 and entitled “Digital Microfluidic Apparatus and Testing Method Thereof”, which are hereby incorporated herein by reference in their entireties.

### TECHNICAL FIELD

The present disclosure relates to, but is not limited to, the field of micro-electromechanical technologies, in particular to a digital microfluidic device, a drive method of the digital microfluidic device and use of the digital microfluidic device.

### BACKGROUND

Microfluidic refers to science and technology involved in a system of processing or manipulating microfluids (nanoliter to attoliter in volume) by using micro-channels (tens to hundreds of micrometers in size), and is a new interdisciplinary subject involving chemistry, fluid physics, microelectronics, new materials, biology, and biomedical engineering. Because of characteristics of miniaturization and integration, etc., a microfluidic device is usually called a microfluidic chip, also known as a lab on chip or a micro total analysis system. One of important characteristics of microfluidic is that it has unique fluid properties in micro-scale environment, such as laminar flow and droplets. With help of these unique fluid phenomena, microfluidic may achieve micromachining and micro-operation that are difficult to be completed by a series of conventional methods.

At present, microfluidic is considered to have great development potential and wide application prospects in biomedical research.

### SUMMARY

The following is a summary of subject matter described herein in detail. The summary is not intended to limit the protection scope of the present disclosure.

In one aspect, an embodiment of the present disclosure provides a drive method of a digital microfluidic device, an array element of the digital microfluidic device having a drive electrode and a reference electrode, the drive method including:
applying a first reference voltage to the reference electrode; and
addressing the drive electrode according to a set of data, including:
(i) when a first scan voltage is applied, a first data voltage is written to a corresponding array drive electrode to define a voltage difference with a magnitude equal to or greater than an actuation voltage between two ends of the array element, and the array element is in an actuated state;
(ii) when a second scan voltage is applied, the first data voltage is electrically isolated from the corresponding array drive electrode to define a voltage difference with a magnitude less than the actuation voltage between the two ends of the array element, the array element is in a non-actuated state;
(iii) alternately writing the first scan voltage and the second scan voltage to the array element to make the array element be alternately in an actuated state and a non-actuated state;
wherein in the actuated state, the array element is configured to actuate a droplet in the array element, and in the non-actuated state, the array element is configured not to actuate the droplet in the array element;
the droplet in the array element is processed into a target droplet having a diameter smaller than a diameter of the droplet by alternately performing actuation and non-actuation processing.

In an exemplary implementation mode, the first scan voltage is a valid level; and the second scan voltage is an invalid level.

In an exemplary implementation mode, the drive method of the digital microfluidic device further includes: forming a composite droplet in the digital microfluidic device;
the array element is made to be alternately in the actuated state and the non-actuated state to make a solid-liquid contact surface at a position where the droplet is located vary between hydrophilic/hydrophobic states.

In an exemplary implementation mode, the drive method of the digital microfluidic device further includes: heating the droplet by using a temperature control module to reduce the diameter of the droplet to obtain the target droplet.

In an exemplary implementation mode, the diameter of the target droplet is less than or equal to 10 μm.

In an exemplary implementation mode, the diameter of the target droplet is 20 μm to 50 μm.

In an exemplary implementation mode, the diameter of the target droplet is less than or equal to 100 μm.

In an exemplary implementation mode, a frequency F at which the drive electrode alternates between turned-on and turned-off is less than or equal to 50 Hz.

In an exemplary implementation mode, a temperature T at which the droplet is heated is less than or equal to 50° C.

In an exemplary implementation mode, treatment time t for processing the composite droplet is less than 1 min.

In another aspect, an embodiment of the present disclosure provides a digital microfluidic device including a digital microfluidic chip, the digital microfluidic chip including at least a drive electrode and a reference electrode;
the reference electrode is configured to write a first reference voltage;
the drive electrode is configured to alternately write a first scan voltage and a second scan voltage so as to be alternately in an actuated state and a non-actuated state, and in the actuated state, the drive electrode is configured to actuate a composite droplet in the digital microfluidic chip, and in the non-actuated state, the drive electrode is configured not to actuate the composite droplet in the digital microfluidic chip;
the composite droplet is processed into a target droplet having a diameter smaller than a diameter of the composite droplet by alternately performing actuation and non-actuation processing.

In an exemplary implementation mode, the digital microfluidic device further includes a temperature control module and a control module, the digital microfluidic chip further includes a reaction zone and a treatment zone, the reaction zone is configured to form the composite droplet, and the treatment zone is configured to process the composite droplet; the temperature control module is configured to provide a set temperature for the treatment zone, the control module is connected to the digital microfluidic chip and the temperature control module, and the control module is configured to control the temperature of the temperature control module and control an operation mode of the digital microfluidic chip to process the composite droplet in the treatment zone into the target droplet.

In an exemplary implementation mode, cell thicknesses of the drive electrode and the digital microfluidic chip satisfy a following formula:

$$\frac{L}{H} = - \sqrt{2} tan θ$$

wherein θ represents an initial contact angle between the droplet and a hydrophobic surface, H represents a cell thickness of the digital microfluidics chip, and L represents a size of the drive electrode.

In an exemplary implementation mode, the diameter of the target droplet is less than or equal to 10 μm.

In an exemplary implementation mode, the diameter of the target droplet is 20 μm to 50 μm.

In an exemplary implementation mode, the diameter of the target droplet is less than or equal to 100 μm.

In an exemplary implementation mode, the cell thickness H of the digital microfluidics chip is less than or equal to 10 μm, and the size L of the drive electrode is less than or equal to 12.25 μm.

In an exemplary implementation mode, the cell thickness H of the digital microfluidics chip is less than or equal to 10 μm to 30 μm, and the size L of the drive electrode is less than or equal to 12 μm to 50 μm.

In an exemplary implementation mode, the cell thickness H of the digital microfluidics chip is 30 μm to 200 μm, and the size L of the drive electrode is 50 μm to 2 mm.

In an exemplary implementation mode, the operation mode of the digital microfluidic chip is as follows: the drive electrode is controlled by the control module to alternate between turned-on and turned-off to make a solid-liquid contact surface at the position where the droplet is located vary between hydrophilic/hydrophobic states during heating.

In another aspect, an embodiment of the present disclosure further provides a detection method employing the aforementioned digital microfluidic device, including:
forming a composite droplet in the reaction zone of the digital microfluidic chip; and
controlling the drive electrode of the digital microfluidic chip by the control module to alternate between turned-on and turned-off to make a solid-liquid contact surface at the position where the droplet is located vary between hydrophilic/hydrophobic states during heating, to process the composite droplet into the target droplet with the diameter less than or equal to 10 μm; and
a frequency F at which the drive electrode alternates between turned-on and turned-off is less than or equal to 50 Hz, and treatment time t for processing the composite droplet is less than 1 min.

In another aspect, an embodiment of the present disclosure further provides a method for screening a single cell employing the aforementioned digital microfluidic device, including:
forming a droplet containing the single cell in the reaction zone of the digital microfluidic chip, and at least part of the droplet contains the single cell;
driving the droplet by the drive electrode to move to the treatment zone for processing;
controlling the drive electrode located in the treatment zone by the control module to alternate between turned-on and turned-off to make a solid-liquid contact surface at the position where the droplet is located vary between hydrophilic/hydrophobic states during heating, thereby reducing the diameter of the droplet to 20 μm to 50 μm; the droplet with reduced diameter including a target droplet containing at most one of the single cell;
screening out a droplet containing the single cell using optical difference of the target droplet;
wherein a frequency F at which the drive electrode alternates between turned-on and turned-off is less than or equal to 50 Hz, and treatment time t for processing the droplet is less than 1 min.

In another aspect, an embodiment of the present disclosure further provides a library creation and detection method employing the aforementioned digital microfluidic device, including:
forming a composite droplet containing a library in the reaction zone of the digital microfluidic chip;
driving the composite droplet by the drive electrode to move to the treatment zone for processing;
controlling the drive electrode located in the treatment zone by the control module to alternate between turned-on and turned-off to make a solid-liquid contact surface at the position where the composite droplet is located vary between hydrophilic/hydrophobic states, thereby reducing the diameter of the composite droplet to less than or equal to 100 μm;
detecting nucleic acid content and quality of the target droplet by using optical difference of the target droplet, thereby obtaining the nucleic acid content and the quality of the composite droplet;
wherein, a frequency F at which the drive electrode alternates between turned-on and turned-off is less than or equal to 50 Hz, and treatment time t for processing the droplet is less than 1 min.

In an exemplary implementation mode, the method further includes controlling a temperature T of the temperature control module to be less than or equal to 50° C. by the control module.

Other aspects may be comprehended after the drawings and the detailed descriptions are read and understood.

### BRIEF DESCRIPTION OF DRAWINGS

Accompanying drawings are intended to provide further understanding of technical solutions of the present disclosure and constitute a part of the specification, are intended to explain the technical solutions of the present disclosure together with embodiments of the present application, and do not constitute limitations on the technical solutions of the present disclosure. Shapes and sizes of various components in the drawings do not reflect actual scales, but are only intended to schematically illustrate contents of the present disclosure.

FIG. 1 is a schematic diagram of a structure of a digital microfluidic device according to an exemplary embodiment of the present disclosure.

FIG. 2 is a schematic diagram of a planar structure of a digital microfluidics chip employed by a digital microfluidic device according to an exemplary embodiment of the present disclosure.

FIG. 3 is a schematic diagram of a longitudinal sectional structure of a digital microfluidics chip according to an exemplary embodiment of the present disclosure.

FIG. 4 is a schematic diagram of distribution of drive electrodes of a digital microfluidic chip according to an exemplary embodiment of the present disclosure.

FIG. 5 is a schematic diagram of a sectional structure of a digital microfluidics chip employed in a digital microfluidic device according to an exemplary embodiment of the present disclosure.

FIG. 6 is a schematic diagram of a structure of another digital microfluidic device according to an exemplary embodiment of the present disclosure.

FIG. 7 is a schematic diagram of a structure of another digital microfluidic device according to an exemplary embodiment of the present disclosure.

FIG. 8 is a schematic diagram of a principle of an incubation process of a sample liquid according to an exemplary embodiment of the present disclosure.

FIG. 9 is a schematic diagram of a preparation process of a sample to be detected according to an exemplary embodiment of the present disclosure.

FIG. 10 is a top view of droplet array after volume reduction of sample by thermal evaporation according to an exemplary embodiment of the present disclosure.

FIG. 11 is a schematic diagram of a droplet in a digital microfluidic chip according to an exemplary embodiment of the present disclosure.

FIG. 12 is a fluorescence image of a reaction system of a sample to be detected according to an exemplary embodiment of the present disclosure.

FIG. 13A is a schematic diagram of a droplet obtained by a conventional heating method.

FIG. 13B is a schematic diagram of a droplet obtained employing a heating method according to an exemplary embodiment of the present disclosure.

FIG. 14 is a schematic diagram of two-factor joint detection fluorescence images according to an exemplary embodiment of the present disclosure.

FIG. 15 is a standard curve of an effective fluorescence coding bead VS standard concentration according to an exemplary embodiment of the present disclosure.

### DETAILED DESCRIPTION

Specific implementation modes of the present disclosure will be described further in detail below with reference to the accompanying drawings and embodiments. Following embodiments serve to illustrate the present disclosure, but are not intended to limit the scope of the present disclosure. It should be noted that the embodiments in the present application and features in the embodiments may be randomly combined with each other if there is no conflict.

To make the objectives, technical solutions, and advantages of the present disclosure clearer, the embodiments of the present disclosure will be described in detail below in combination with the accompany drawings. It is to be noted that the implementation modes may be implemented in various forms. Those of ordinary skills in the art can easily understand such a fact that implementation modes and contents may be transformed into various forms without departing from the purpose and scope of the present disclosure. Therefore, the present disclosure should not be explained as being limited to the contents recorded in the following implementation modes only. The embodiments and features in the embodiments of the present disclosure may be randomly combined with each other if there is no conflict.

Scales of the drawings in the present disclosure may be used as a reference in actual processes, but are not limited thereto. For example, a width-length ratio of a channel, a thickness and spacing of each film layer, and a width and spacing of each signal line may be adjusted according to actual needs. A quantity of pixels in a display substrate and a quantity of sub-pixels in each pixel are not limited to numbers shown in the drawings. The drawings described in the present disclosure are schematic structural diagrams only, and one implementation mode of the present disclosure is not limited to shapes, numerical values, or the like shown in the drawings.

Ordinal numerals “first”, “second”, “third”, etc., in the specification are set not to form limitations in number but only to avoid the confusion of composition elements.

In the specification, for convenience, expressions “central”, “above”, “below”, “front”, “back”, “vertical”, “horizontal”, “top”, “bottom”, “inside”, “outside”, etc., indicating directional or positional relationships are used to illustrate positional relationships between the composition elements, not to indicate or imply that involved devices or elements are required to have specific orientations and be structured and operated with the specific orientations but only to easily and simply describe the present specification, and thus should not be understood as limitations on the present disclosure. The positional relationships between the composition elements may be changed as appropriate according to the direction according to which each composition element is described. Therefore, appropriate replacements based on situations are allowed, which are not limited to the expressions in the specification.

In the specification, unless otherwise specified and defined, terms “mounting”, “mutual connection”, and “connection” should be generally understood. For example, the term may be fixed connection, or detachable connection, or an integral connection. The term may be mechanical connection or electrical connection. The term may be direct connection, or indirect connection through an intermediate, or internal communication between two elements. Those of ordinary skills in the art can understand specific meanings of the above terms in the present disclosure according to specific situations.

In the specification, a transistor refers to an element that at least includes three terminals, i.e., a gate electrode, a drain electrode, and a source electrode. The transistor has a channel region between the drain electrode (drain electrode terminal, drain region, or drain electrode) and the source electrode (source electrode terminal, source region, or source electrode), and a current can flow through the drain electrode, the channel region, and the source region. It is to be noted that in the specification, the channel region refers to a region through which a current mainly flows through.

In the specification, “electrical connection” includes connection of composition elements through an element with a certain electrical action. “An element with a certain electrical action” is not particularly limited as long as electric signals between the connected composition elements may be sent and received. Examples of the “element with the certain electrical action” not only include an electrode and a line, but also include a switch element such as a transistor, a resistor, an inductor, a capacitor, or another element with various functions, etc.

In the specification, “parallel” refers to a state in which an angle formed by two straight lines is −10° or more and 10° or less, and thus also includes a state in which the angle is −5° or more and 5° or less. In addition, “perpendicular” refers to a state in which an angle formed by two straight lines is 80° or more and 100° or less, and thus also includes a state in which the angle is 85° or more and 95° or less.

Triangle, rectangle, trapezoid, pentagon, hexagon, etc. in this specification are not strictly defined, and they may be approximate triangle, rectangle, trapezoid, pentagon, hexagon, etc. There may be some small deformations caused by tolerance, and there may be chamfer, arc edge, deformation, etc.

In the present disclosure, “about” refers to that a boundary is defined not so strictly and numerical values within process and measurement error ranges are allowed.

A digital microfluidics chip uses a principle of Electrowetting on Dielectric (EWOD), a droplet is disposed on a surface having a hydrophobic layer, with help of an electrowetting effect, wettability between the droplet and the hydrophobic layer is changed by applying a voltage to the droplet, so that a pressure difference and asymmetric deformation are generated inside the droplet, furthermore, directional movement of the droplet may be achieved, and the droplet may be moved, mixed, and separated at micron scale. It has a capability to miniaturize basic functions of a biological laboratory, a chemical laboratory, etc. to a chip of several square centimeters, and has advantages of a small size, portability, flexible combination of functions, a high integration degree, etc.

Digital microfluidics is divided into active digital microfluidics and passive digital microfluidics. A main difference between them is that active digital microfluidics is to drive droplets in an array mode, which may accurately control a droplet at a certain position to move separately, while passive digital microfluidics is that droplets at all positions are moved or stopped together. The active digital microfluidics technology may achieve independent control of drive electrodes by setting Thin Film Transistor (TFT for short) to control the drive electrodes, thus achieving accurate control of the droplets. Compared with the passive digital microfluidics technology, for M×N drive electrodes, the passive digital microfluidic technology needs M×N control signals, while the active digital microfluidics technology only needs M+N control signals with the help of its drive mode of row addressing and column addressing, wherein M and N are positive integers greater than 1. Therefore, active digital microfluidics is more suitable for manipulating high-throughput samples, and may achieve arbitrary programmable movement paths of single/multiple droplet(s), and multiple samples may be manipulated simultaneously and in parallel. Process flows of the active digital microfluidics technology may be compatible with fabrication of electrical and optical sensors, and allow electrical detection, optical detection, and other means to be integrated into a chip to form a multi-functional active digital microfluidics chip laboratory. In recent years, the digital microfluidic chip, as a new technology for micro fluid operation and control, has shown great development potential and application prospects in the fields of biology, chemistry, medicine, due to its advantages such as simple structure, small required amount of sample and reagent, easiness in integration, parallel treatment, and easiness in automation.

Immunoassay is a physiological function detection in which a body recognizes “autoantigen” and “non-self” antigens, forms natural immune tolerance to autoantibodies, and rejects “non-self” antigens. Under normal circumstances, this physiological function is beneficial to the body, which can produce immune protection effects such as anti-infection, anti-tumor and the like to maintain physiological balance and stability of the body. Under certain conditions, when the immune function is dysfunctional, it will also produce harmful reactions and results to the body, which are often manifested as various immune diseases in clinic, such as immunodeficiency diseases, autoimmune diseases, bacterial invasion, virus infection and tumors.

The traditional ELISA (Enzyme Linked Immunosorbent Assay) technique is one of the effective immunoassay techniques, its detection sensitivity is about 10 pg/mL, however one index can only be detected from one sample, thus some low-abundance protein molecular can not be detected by ELISA, which has long been unable to meet the clinical needs. In order to improve the sensitivity of immunoassay, a chemiluminescence detection technology has been developed by in vitro diagnostic manufacturers, which has a sensitivity 10 to 100 times higher than that of the traditional ELISA, and has become the most important detection technology in the current immunodiagnosis market. Since the appearance of chemiluminescence technology in 1970s, although detection sensitivity of the chemiluminescence technology has been significantly improved with the development of full automation level of detection equipment and precision of detection elements, in essence, the chemiluminescence technology in detection principle has not changed over the past 50 years. It can be considered that chemiluminescence technology has approached its limit of detection ability, and the highest sensitivity can be up to 1 pg/mL.

Single molecule immunoassay refers to detection of protein molecules of a single molecule level implemented by capturing and recognizing antigen molecules with antibodies, and by single molecule fluorescence signal detection or single molecule enzymatic reaction, and its detection sensitivity far exceeds the existing chemiluminescence technology platform. At present, the only commercialized technical platforms for single molecule-level immunoassay in the world are SiMoA system of Quanterix Company and SMC system of Merck. SiMoA system and SMC system represent two technical strategies for realizing single molecule immunoassay under existing technology, namely, reducing sensitivity requirement of detection equipment in a manner of signal amplification and implementing molecular-level counting in a manner of improving the sensitivity of the detection equipment. Among the two strategies, the former has complex reagent operation process, difficult equipment automation, high cost of chip consumables and poor system stability, while the latter has difficult calibration of optical inspection equipment, easy blockage of liquid channel and easy environmental interference. Although the detection sensitivity of the two systems is far higher than that of the current mainstream chemiluminescence technology platforms, other characteristics are far from meeting requirements of medical diagnostic products.

In addition, the digital microfluidic chip technology can also be used in the technical field of single cell detection, for example, hybridoma single cell detection in a process of producing monoclonal antibody drugs, etc.; and used in the technical field of Polymerase Chaim Reaction (PCR), for example, preparing PCR amplification library, etc. How to apply microfluidic technology to the technical fields of single molecule immunoassay, single cell detection, PCR amplification library and the other, to achieve multi-indexes and high sensitivity detection of rare and low abundance samples by automatic and rapid detection process, has great significance.

An exemplary embodiment of the present disclosure provides a drive method of a digital microfluidic device, an array element of the digital microfluidic device having drive electrodes and a reference electrode, the drive method including:
applying a first reference voltage to the reference electrode; and
addressing the drive electrode according to a set of data, including:
(i) when a first scan voltage is applied, a first data voltage is written to a corresponding drive electrode in the array to define a voltage difference with a magnitude equal to or greater than an actuation voltage between two ends of the array element, and the array element is in an actuated state;
(ii) when a second scan voltage is applied, the first data voltage is electrically isolated from the corresponding drive electrode in the array to define a voltage difference with a magnitude less than the actuation voltage between the two ends of the array element, the array element is in a non-actuated state;
(iii) alternately writing the first scan voltage and the second scan voltage to the array element to make the array element be alternately in an actuated state and a non-actuated state;
wherein in the actuated state, the array element is configured to actuate a droplet in the array element, and in the non-actuated state, the array element is configured not to actuate the droplet in the array element;
the droplet in the array element is processed into a target droplet having a diameter smaller than a diameter of the droplet by alternately performing actuation and non-actuation processing.

After processing, the diameter of the droplet is reduced to make the droplet be more solid, and substances in the droplet are more evenly distributed and concentrated, which can enhance signal quantity of signals (e.g., optical signals) as they pass through the droplet.

In an exemplary implementation mode, the first scan voltage is a valid level; and the second scan voltage is an invalid level.

In an exemplary implementation mode, the drive method of the digital microfluidic device further includes: forming a droplet in the digital microfluidic device; and
the array element is made to be alternately in the actuated state and the non-actuated state to make a solid-liquid contact surface at a position where the droplet is located vary between hydrophilic/hydrophobic states.

In an exemplary implementation mode, the drive method of the digital microfluidic device further includes: reducing the diameter of the droplet using natural evaporation to make the droplet be processed into the target droplet.

In an exemplary implementation mode, the drive method of the digital microfluidic device further includes: heating the droplet by using a temperature control module to reduce the diameter of the droplet to obtain a target droplet.

In an exemplary implementation mode, the diameter of the target droplet is less than or equal to 10 μm.

In an exemplary implementation mode, the diameter of the target droplet is 20 μm to 50 μm.

In an exemplary implementation mode, the diameter of the target droplet is less than or equal to 100 μm.

In an exemplary implementation mode, a frequency F at which the drive electrode alternates between turned-on and turned-off is less than or equal to 50 Hz.

In an exemplary implementation mode, a temperature T at which the droplet is heated is less than or equal to 50° C.

For example, when the drive method is used in a single molecule detection process, the diameter of the target droplet may be less than or equal to 10 μm, the frequency F at which the drive electrode alternates between turned-on and turned-off is less than or equal to 50 Hz, and the temperature T at which the droplet is heated is less than or equal to 50° C.

For example, when the drive method is used in the single cell detection process, the diameter of the target droplet may be 20 μm to 50 μm, the frequency F at which the drive electrode alternates between turned-on and turned-off is less than or equal to 50 Hz, and the temperature T at which the droplet is heated is less than or equal to 50° C.

For example, when the drive method is used in a PCR library creation process, the diameter of the target droplet may be less than or equal to 100 μm, the frequency F at which the drive electrode alternates between turned-on and turned-off is less than or equal to 50 Hz, and the temperature T at which the droplet is heated is less than or equal to 50° C.

In an exemplary implementation mode, the treatment time t for processing the droplet is less than 1 min.

An exemplary embodiment of the present disclosure further provides a digital microfluidic device including a digital microfluidic chip, the digital microfluidic chip including at least a drive electrode and a reference electrode;
the reference electrode is configured to write a first reference voltage;
the drive electrode is configured to alternately write a first scan voltage and a second scan voltage so as to be alternately in an actuated state and a non-actuated state, and in the actuated state, the drive electrode is configured to actuate a composite droplet present in the digital microfluidic chip, and in the non-actuated state, the drive electrode is configured not to actuate the composite droplet in the digital microfluidic chip; and
the composite droplet is processed into a target droplet having a diameter smaller than a diameter of the composite droplet by alternately performing actuation and non-actuation processing.

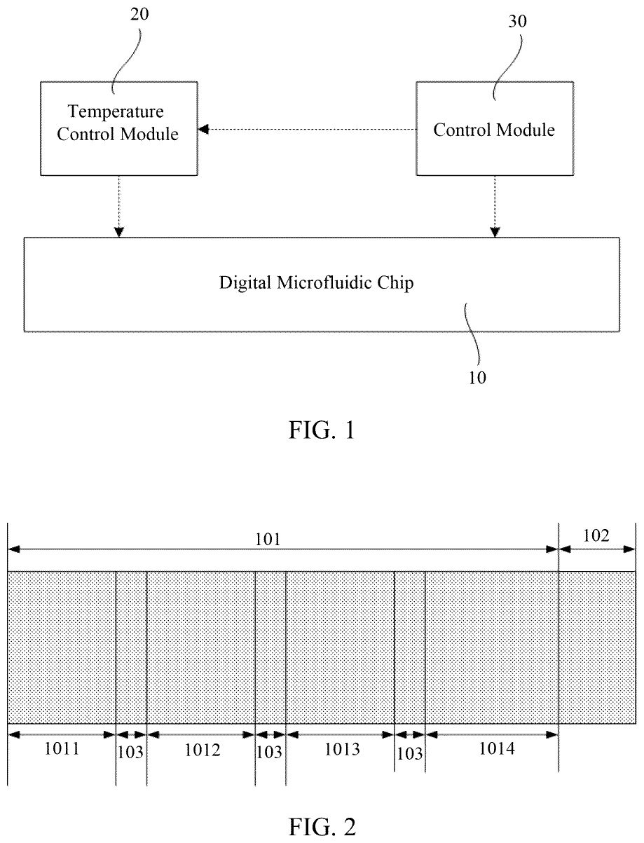

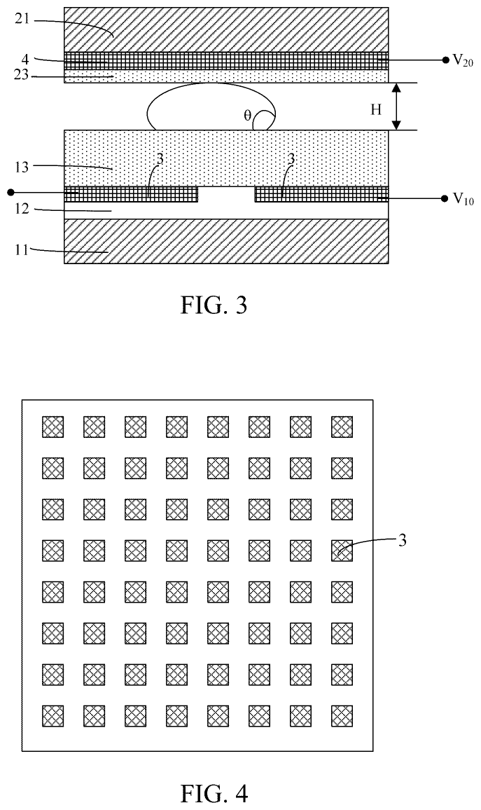

FIG. 1 is a schematic diagram of a structure of a digital microfluidic device according to an exemplary embodiment of the present disclosure, FIG. 2 is a schematic diagram of a planar structure of a digital microfluidics chip according to an exemplary embodiment of the present disclosure, FIG. 3 is a schematic diagram of a longitudinal sectional structure of a digital microfluidics chip according to an exemplary embodiment of the present disclosure, and FIG. 4 is a schematic diagram of the distribution of drive electrodes of a digital microfluidic chip according to an exemplary embodiment of the present disclosure.

As shown in FIGS. 1 and 2, the digital microfluidic device may at least include a digital microfluidic chip 10, a temperature control module 20 and a control module 30. The digital microfluidic chip 10 may at least include a reaction zone 101 and a treatment zone 102. The reaction zone 101 is configured to form composite droplets and the treatment zone 102 is configured to process the composite droplets. The temperature control module 20 is configured to provide a set temperature for the treatment zone 102. The control module 30 is connected to the digital microfluidic chip 10 and the temperature control module 20, and the control module 30 is configured to control a temperature of the temperature control module 20 and to control an operation mode of the digital microfluidic chip 10 so that the composite droplets in the treatment zone 102 are processed into target droplets having a diameter smaller than that of the composite droplets.

As shown in FIGS. 3 and 4, the digital microfluidic chip 10 further includes drive electrodes 3 and a reference electrode 4, and the electrodes 3 are distributed in an array. In the digital microfluidic chip 10 as shown in FIG. 3, the drive electrode 3 applies a first scan voltage or a second scan voltage through V10, and the reference electrode 4 applies a first reference voltage through V20.

In an exemplary embodiment, a diameter of a target droplet may be less than or equal to 10 μm.

In an exemplary embodiment, the diameter of the target droplet may be 20 μm to 50 μm.

In an exemplary embodiment, the diameter of the target droplet may be less than or equal to 100 μm. In an exemplary embodiment, as shown in FIG. 2, the reaction zone 101 may at least include a first mix incubation zone 1011, a second mix incubation zone 1012, a third mix incubation zone 1013, and a composite droplet formation zone 1014 which are sequentially communicated. Among them, the first mix incubation zone 1011 is configured to enable binding between fluorescence coding magnetic beads and capture antibodies to form a magnetic bead antibody. The second mix incubation zone 1012 is configured to enable binding between the magnetic bead antibody and a target molecule to form a fluorescence coding magnetic bead-capture antibody-target molecule conjugate. The third mix incubation zone 1013 is configured to enable binding between the fluorescence coding magnetic bead-capture antibody-target molecule conjugate and an enzyme label detection antibody conjugate to form a fluorescence coding magnetic bead-capture antibody-target molecule conjugate-enzyme label detection antibody conjugate. The composite droplet formation zone 1014 is configured to enable mixing of the fluorescence coding magnetic bead-capture antibody-target molecule conjugate-enzyme label detection antibody conjugate with a fluorescent substrate to form composite droplets.

In an exemplary embodiment, the first mix incubation zone 1011 and the second mix incubation zone 1012 are communicated by a purification channel 103, the second mix incubation zone 1012 and the third mix incubation zone 1013 are communicated by a purification channel 103, and the third mix incubation zone 1013 and the composite droplet formation zone are communicated by a purification channel 103.

In an exemplary embodiment, the control module 30 may also be configured to drive and manipulate a path of a droplet in the digital microfluidic chip, enabling programmable path manipulation of the droplet.

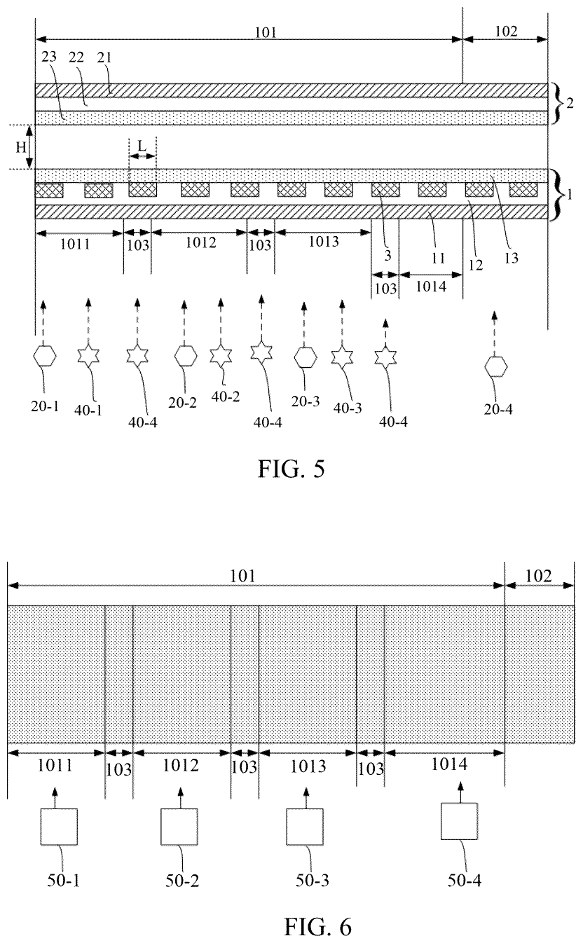

FIG. 5 is a schematic diagram of a sectional structure of a digital microfluidics chip according to an exemplary embodiment of the present disclosure. As shown in FIG. 5, in an exemplary implementation mode, the digital microfluidics chip 10 may include a first substrate 1 and a second substrate 2 disposed oppositely, the first substrate 1 may at least include a first base substrate 11, a first structure layer 12 disposed on a side of the first base substrate 11 facing the second substrate 2, and a first lyophobic layer 13 disposed on a side of the first structure layer 12 facing the second substrate 2. The second substrate 2 may include a second base substrate 21, a second structure layer 22 disposed on a side of the second base substrate 21 facing the first substrate 1, and a second lyophobic layer 23 disposed on a side of the second structure layer 22 facing the first substrate.

In an exemplary implementation mode, the first substrate 1 and the second substrate 2 disposed oppositely may be cell-assembled by a sealant, the first substrate 1, the second substrate 2, and the sealant together form a closed treatment cavity and a sample to be treated may be disposed in the treatment cavity. In an exemplary embodiment, the treatment cavity may be divided into a plurality of functional zones sequentially disposed, and the plurality of functional zones may at least include a reaction zone 101 and a treatment zone 102 in communication with the reaction zone 101. The reaction zone 101 is configured to form a composite droplet and the treatment zone 102 is configured to treat the composite droplet.

In an exemplary embodiment, a plurality of drive electrodes 3 disposed in an array are provided corresponding to the reaction zone 101 and the treatment zone 102, and a cell thickness of the drive electrodes 3 and the digital microfluidic chip 10 satisfies a following formula:

$$\frac{L}{H} = - \sqrt{2} tan θ$$

Herein, θ represents an initial contact angle between a droplet and a hydrophobic surface, and θ is generally close to 120°, H represents a cell thickness of a digital microfluidics chip, and L represents a size of a drive electrode 3.

In an exemplary embodiment, the drive electrodes 3 are disposed in the first structural layer 12 of the digital microfluidic chip 10.

In an exemplary implementation mode, the cell thickness H of the digital microfluidics chip 10 is less than or equal to 10 μm, and the size L of each drive electrode 3 is less than or equal to 12.25 μm.

In an exemplary implementation mode, the cell thickness H of the digital microfluidics chip 10 is 10 μm to 30 μm, and the size L of each drive electrode 3 is 12 μm to 50 μm.

In an exemplary implementation mode, the cell thickness H of the digital microfluidics chip 10 is 30 μm to 200 μm, and the size L of each drive electrode 3 is 50 μm to 2 mm.

In an exemplary embodiment, the diameter of the target droplet is less than or equal to 10 μm, the cell thickness H of the digital microfluidic chip 10 is less than or equal to 10 μm, and the size L of each drive electrode 3 is less than or equal to 12.25 μm.

In an exemplary embodiment, the diameter of the target droplet is 20 μm to 50 μm, the cell thickness H of the digital microfluidic chip 10 is 10 μm to 30 μm, and the size L of each drive electrode 3 is 12 μm to 50 μm.

In an exemplary embodiment, the diameter of the target droplet is less than or equal to 100 μm, the cell thickness H of the digital microfluidic chip 10 is 30 μm to 200 μm, and the size L of each drive electrode 3 is 50 μm to 2 mm.

In an exemplary embodiment, an operation mode of the digital microfluidic chip is as follows: the drive electrode 3 is controlled to alternate between turned-on (ON) and turned-off (OFF) so that the composite droplets placed in the treatment zone 102 vary between hydrophilic/hydrophobic states during heating.

In an exemplary embodiment, a frequency F for alternating between turned-on (ON) and turned-off (OFF) is less than or equal to 50 Hz.

In an exemplary embodiment, the treatment zone provides a set temperature less than or equal to 50° C.

In an exemplary embodiment, treatment time t is less than 1 min.

In an exemplary embodiment, the plurality of drive electrodes 3 may be divided into a plurality of units corresponding to the reaction zone and the treatment zone to form a reaction zone drive unit and a treatment zone drive unit. An operation mode of the digital microfluidic chip is as follows: drive electrodes in the treatment zone drive unit are controlled to alternate between turned-on (ON) and turned-off (OFF), so that composite droplets placed in the treatment zone 102 vary between hydrophilic/hydrophobic states during heating, wherein a frequency F for alternating turned-on (ON) and turned-off (OFF) is less than or equal to 50 Hz, the treatment zone provides a set temperature less than or equal to 50° C., and treatment time t is less than 1 min.

In an exemplary embodiment, the reaction zone drive unit may be at least divided into a first reaction zone drive unit, a second reaction zone drive unit, a third reaction zone drive unit, and a fourth reaction zone drive unit which are respectively corresponding to the first mix incubation zone 1011, the second mix incubation zone 1012, the third mix incubation zone 1013, and the composite droplet formation zone 1014, respectively. An operation mode of the digital microfluidic chip is as follows: drive electrodes in the first reaction zone drive unit, the second reaction zone drive unit, the third reaction zone drive unit and the fourth reaction zone drive unit are controlled to provide required driving states for droplets in the corresponding functional zones. In an exemplary embodiment, the digital microfluidic chip further includes a drive transistor connected to the drive electrode 3 and the control module 30, and the drive electrode 3 is controlled by the control module 30 through the drive transistor.

In an exemplary embodiment, as shown in FIG. 5, the temperature control module 20 may include a plurality of sub-modules that implement temperature control functions, which at least include a first temperature control sub-module 20-1 corresponding to the first mix incubation zone 1011, a second temperature control sub-module 20-2 corresponding to the second mix incubation zone 1012, a third temperature control sub-module 20-3 corresponding to the third mix incubation zone 1013, and a fourth temperature control sub-module 20-4 corresponding to the treatment zone 102. The above-mentioned temperature control sub-modules may be disposed on a side of the first substrate 1 away from the second substrate 2, or disposed on a side of the second substrate 2 away from the first substrate 1, corresponding to respective functional zones, and provide suitable temperatures for the corresponding functional zones, respectively.

In an exemplary embodiment, the control module 30 is at least configured to control a temperature of the fourth temperature control sub-module 20-4 and to control the operation mode of the digital microfluidic chip 10 such that the composite droplets in the treatment zone 102 are processed into target droplets with a droplet diameter less than or equal to 10 μm.

In an exemplary embodiment, as shown in FIG. 5, the digital microfluidic device further includes a magnetic control module for generating a magnetic force with a certain field strength, and droplets may be adsorbed and aggregated and get close to a surface of the digital microfluidic chip 10 by using the magnetic control module. The magnetic control module at least includes a first magnetic control sub-module 40-1 corresponding to the first mix incubation zone 1011, a second magnetic control sub-module 40-2 corresponding to the second mix incubation zone 1012, a third magnetic control sub-module 40-3 corresponding to the third mix incubation zone 1013, and a plurality of fourth magnetic control sub-modules 40-4 corresponding to a plurality of purification channels respectively. The above-mentioned magnetic control sub-modules may be disposed on the side of the first substrate 1 away from the second substrate 2, or disposed on the side of the second substrate 2 away from the first substrate 1, which are corresponding to respective functional zones and purification channels, and provide suitable magnetic forces for the corresponding functional zones and purification channels respectively.

FIG. 6 is a schematic diagram of a structure of another digital microfluidic device according to an exemplary embodiment of the present disclosure. In an exemplary embodiment, as shown in FIG. 6, the digital microfluidic device may further include a sample addition module configured to add a sample, reagent, or the like forming a composite droplet to a corresponding zone of the digital microfluidic chip. The sample addition module may at least include a first sample addition sub-module 50-1 corresponding to the first mix incubation zone 1011, a second sample addition sub-module 50-2 corresponding to the second mix incubation zone 1012, a third sample addition sub-module 50-3 corresponding to the third mix incubation zone 1013, and a fourth sample addition sub-module 50-4 corresponding to the composite droplet formation zone 1014. The above-mentioned sample addition sub-modules may be disposed on the first substrate 1 or the second substrate 2, corresponding to a corresponding functional zone. A sample addition port is disposed in each functional zone of the digital microfluidic chip 10 corresponding to each sample addition sub-module, and quantity, position, and size of sample addition ports, and types of sample, solution, and reagent injected into the sample addition port of each functional zone may be set according to actual needs. A desired sample, solution, reagent, etc. are added by the sample addition module 50 to a corresponding functional zone through the sample addition port disposed in each functional zone.

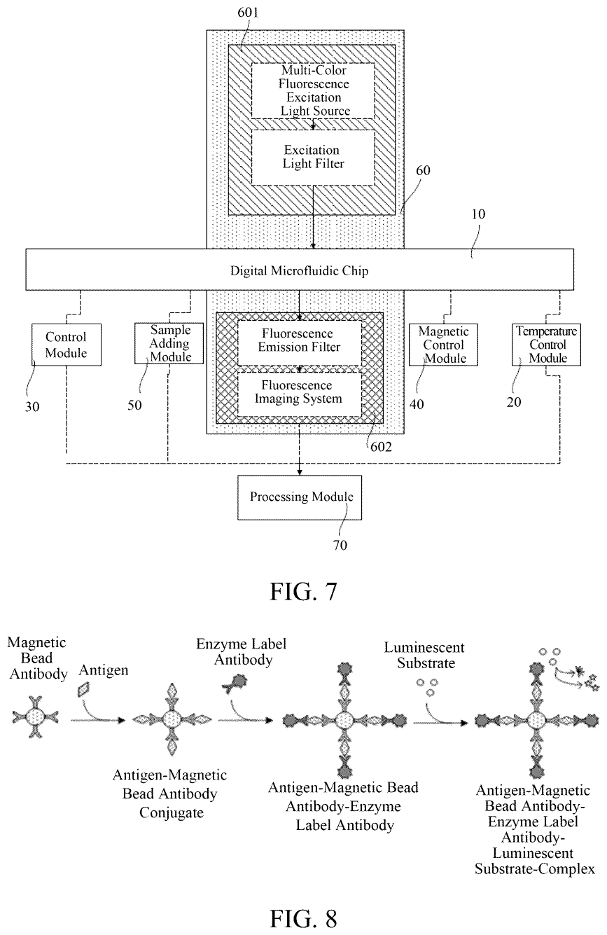

FIG. 7 is a schematic diagram of a structure of another digital microfluidic device according to an exemplary embodiment of the present disclosure. In an exemplary embodiment, the digital microfluidic device may at least include a digital microfluidic chip 10, a temperature control module 20, a control module 30, a magnetic control module 40, a sample addition module 50, and a signal detection module 60. The signal detection module 60 may at least include a fluorescence excitation module 601 providing a light source of a desired wavelength, and a fluorescence imaging module 602 for imaging fluorescence. The fluorescence excitation module 601 is disposed on a side of the digital microfluidic chip and includes a multi-color fluorescence excitation light source and an excitation light filter connected to the multi-color fluorescence excitation light source, and the fluorescence imaging module 602 is disposed on a side of the digital microfluidic chip away from the fluorescence excitation module 601 and includes a fluorescence emission filter and a fluorescence imaging system connected to the fluorescence emission filter.

In an exemplary embodiment, the fluorescence excitation module 601 and fluorescence imaging module 602 are intended to achieve fluorescence detection of a target droplet, and the fluorescence excitation module 601 and fluorescence imaging module 602 may be disposed on two sides or on a same side of the digital microfluidic chip 10 or at other positions respectively, which are not limited herein.

In an exemplary embodiment, the digital microfluidic device may further include a processing module 70, the processing module 70 is connected to a fluorescence imaging module 602 for reading signals generated by the fluorescence imaging module 602 and analyzing and processing the signals, to obtain concentration information. In an exemplary embodiment, the processing module 70 may be a processor or the like.

FIG. 8 is a schematic diagram of a principle of sample liquid incubation process according to an exemplary embodiment of the present disclosure. As shown in FIG. 8, the single molecule immunoassay proposed in this disclosure is based on the principle of enzyme linked immunosorbent assay. The capture antibody is labeled the fluorescent coding magnetic bead surface (referred to as magnetic bead antibody) in the first mixed incubation zone 1011. The capture antibody can be bound to the target molecule to be detected (such as antigen) in the second mixed incubation zone 1012 to obtain antigen-magnetic bead antibody conjugate. The antigen-magnetic bead antibody conjugate is bound to enzyme label detection antibody (referred to as enzyme label antibody) in the third mixed incubation zone 1013 to form antigen-magnetic bead antibody-enzyme label antibody conjugate, then a luminescent substrate is added, and fluorescence signals are emitted from the substrate under the catalysis of enzyme molecules.

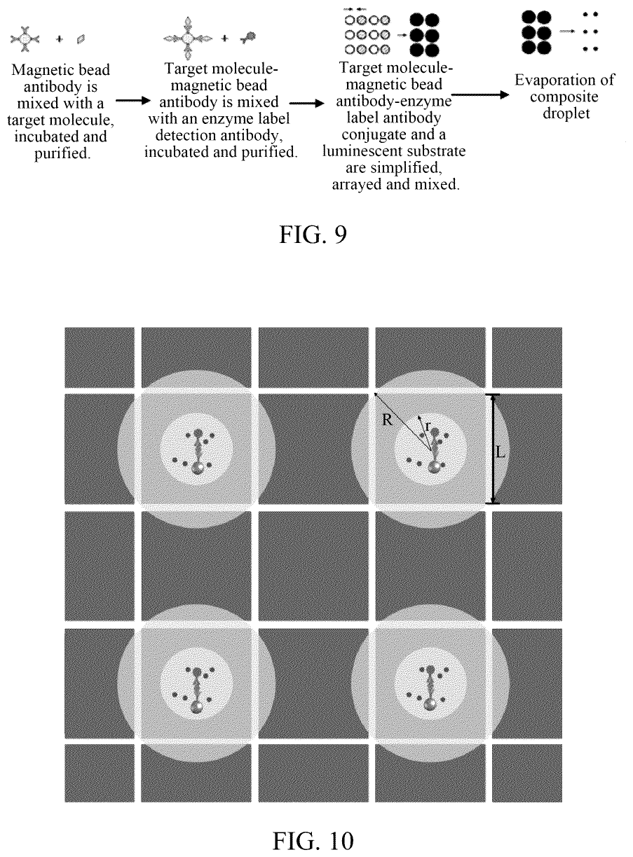

FIG. 9 is a schematic diagram of a preparation process of a sample to be detected according to an exemplary embodiment of the present disclosure. FIG. 10 is a top view of droplet array after volume reduction of sample by thermal evaporation according to an exemplary embodiment of the present disclosure. As shown in FIG. 9, fluorescence coding magnetic bead of the captured target molecule (namely the target molecule-magnetic bead antibody) is obtained after the magnetic bead antibody formed by the capture antibody and the fluorescence coding magnetic bead is evenly mixed with the target molecule, incubated and purified. The target molecule-magnetic bead antibody is then evenly mixed with the enzyme label detection antibody, incubated and purified to obtain a target molecule-magnetic bead antibody-enzyme label antibody conjugate. The target molecule-magnetic bead antibody-enzyme label antibody conjugate and the luminescent substrate are simplified, arrayed and mixed, so that the fluorescence coding magnetic bead, the capture antibody, the target molecule, the enzyme label detection antibody and the fluorescent luminescent substrate are enclosed in a droplet with a radius R, and the droplet radius of the reaction system is reduced to r (as shown in FIG. 9) after heating and evaporation.

In an exemplary embodiment, in order to achieve that there is and there only is one target molecule-magnetic bead antibody-enzyme label antibody conjugate (single particle wrapping) or there is not (does not contain) the target molecule-magnetic bead antibody-enzyme label antibody conjugate in each droplet. It is necessary to set the cell thickness of the digital microfluidic chip, and the size of the drive electrode matching the size of the droplet. In the present disclosure, a mass averaging method is used for calculating a size of single cell wrapping, and it is considered that a distribution of cells in a droplet obeys the Poisson distribution law. A diameter D of fluorescence coding magnetic beads is generally between 1 μm to 10 μm under a certain concentration of magnetic bead particle suspension, and when a volume Vdrop of droplet is close to pL level, single particle wrapping can be achieved. Herein, a volume of a single particle wrapping droplet can be expressed as the following formula:

$$\begin{matrix}{V}_{d r o p} = \frac{\sqrt{2} π {L}^{3}}{2 {sin}^{3} θ} ( - cos θ + \frac{1}{3} {cos}^{3} θ ) \\ \frac{L}{H} = - \sqrt{2} tan θ\end{matrix}$$

θ represents an initial contact angle between a droplet and a hydrophobic surface, which is generally close to 120°, L represents a size of a single drive electrode, and H represents the cell thickness of the digital microfluidic chip.

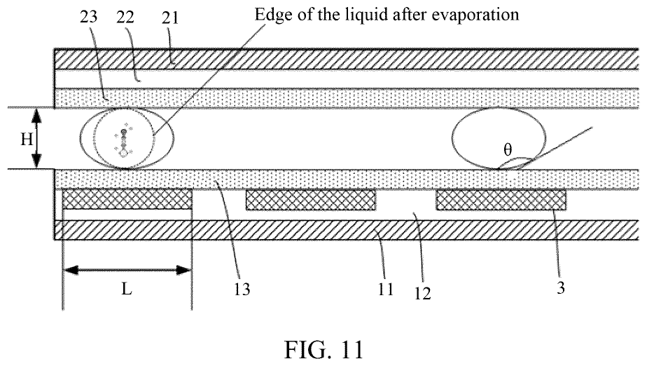

FIG. 11 is a schematic diagram of a droplet in a digital microfluidic chip according to an exemplary embodiment of the present disclosure. As shown in FIG. 11, a cell thickness H of the digital microfluidics chip refers to a distance between a first lyophobic layer 13 in a first substrate 1 and a second lyophobic layer 23 in a second substrate 2, and a size L of a drive electrode refers to a length of the drive electrode along a moving direction of the droplet.

Taking single molecule immunoassay as an example, in order to achieve single cell wrapping, that is, a volume Vdrop of a single droplet is close to pL level, according to the above formula, when the cell thickness H of the digital microfluidics chip is less than or equal to 10 μm, the size L of the single drive electrode is less than or equal to 12.25 μm, and radius R of the single droplet is less than or equal to 13.7 μm.

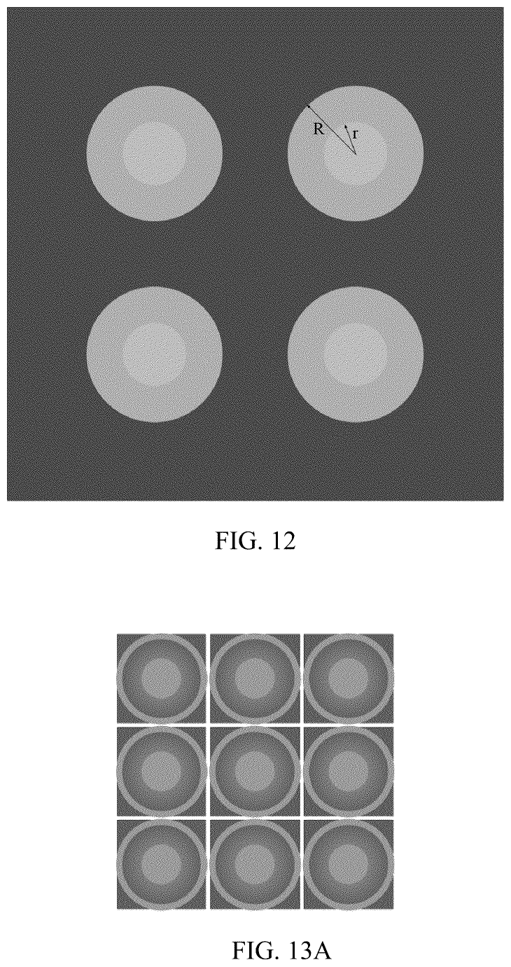

In order to make fluorescence signals of the reaction system more concentrated and improve contrast between a fluorescence signal point and an ambient background, the reaction system is heated, wherein a heating temperature T does not affect normal occurrence of the chemiluminescence reaction. In an exemplary embodiment, T is less than or equal to 50° C., the state of the drive electrode alternates between turned-on (ON) and turned-off (OFF) during heating, and the signal frequency F is less than or equal to 50 Hz, so that the droplets vary between hydrophilic/hydrophobic states during heating, so as to eliminate an edge effect of the contents in the droplet, and make the substance to be detected in the droplet to be aggregated in the center of the droplet. The heating is stopped, when the radius of the droplet to be measured is reduced from R to r during heating, and r is less than or equal to 10 μm. FIG. 12 is a fluorescence image of a reaction system of a sample to be detected according to an exemplary embodiment of the present disclosure, and a radius of a droplet to be detected being reduced from R to r during heating, as shown in FIG. 12.

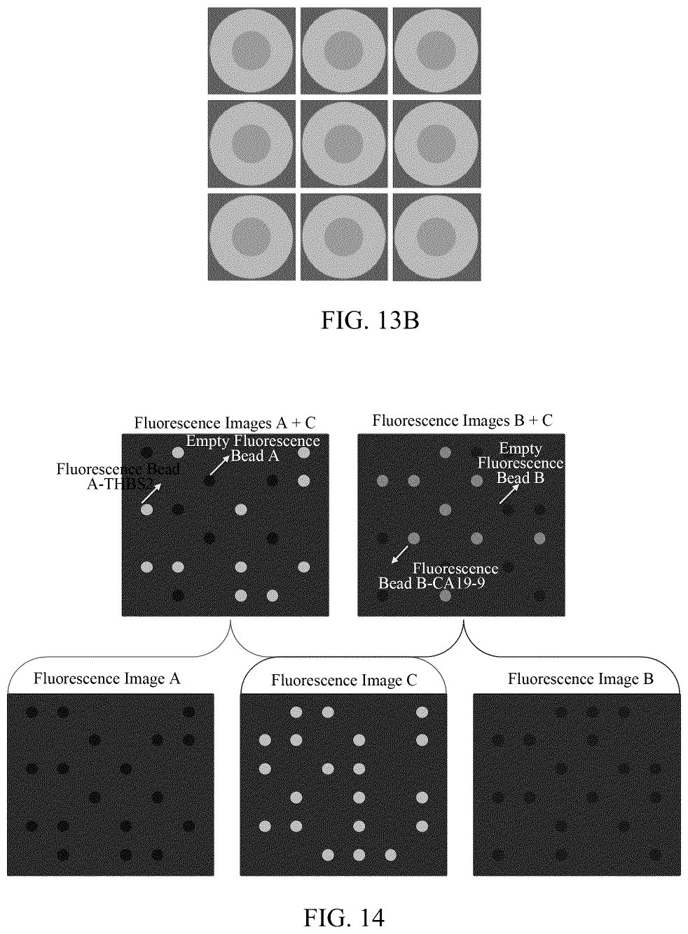

FIGS. 13A and 13B are comparison diagrams of sizes of droplets obtained by different heating methods according to an exemplary embodiment of the present disclosure. In an exemplary embodiment, under same conditions, FIG. 13A shows a droplet obtained by a conventional heating method, and FIG. 13B shows a droplet obtained by employing the heating method of the present disclosure. With the heating method of the present disclosure, a volume of the reaction system can be reduced from pL level to fL level, a signal-to-noise ratio of fluorescence signal can be effectively enhanced, and simultaneous detection with a throughput of more than or equal to 10,000 reaction systems can be finally formed. In an exemplary embodiment, when the target droplet as shown in FIG. 13B is formed, the frequency F at which a drive electrode alternates between turned-on and turned-off is less than or equal to 50 Hz, and the treatment zone 102 provides a set temperature T less than or equal to 50° C., and the time is less than 1 min.

In the present disclosure, automation of a detection process for complex single molecule is achieved by employing a digital microfluidic chip, which includes: a sample to be detected is mixed with a single molecule detection reagent, incubated, purified and a target molecule to be detected is simplified and arrayed; multi-indexes joint detection of a sample can be achieved, using fluorescence coding magnetic bead technology combined with fluorescence imaging technology; automation and rapidity of single molecule immunoassay process can be achieved, and detection of the multi-indexes and high sensitivity of rare and low abundance samples can be achieved, providing powerful tools for fields such as life science research, in vitro diagnosis, concomitant diagnosis and blood screening.

A concentration of target molecules is at fg/ml level and a ratio of target molecule to fluorescence coding magnetic beads is less 1, the fluorescence coding magnetic beads labeled with the target molecule obey the Poisson distribution law. Magnetic beads without labeled with the target molecule do not generate signals. Most of the fluorescence coding magnetic beads labeled with target molecules are labeled by an enzyme molecule, and the fluorescence coding magnetic beads captured with a single target molecule are simplified and arrayed into independent droplets, and the chemiluminescence reaction occurs in fL˜pL droplets, so that single molecule detection of the target molecule can be achieved.

In an exemplary embodiment, a detection method employing a digital microfluidic device for detection is used to detect thrombospondin 2 (THBS2) and carbohydrate protein tumor marker CA19-9 in blood. The above biomarkers are important reference indexes for pancreatic cancer, and detection of concentrations of the thrombospondin 2 and the carbohydrate protein tumor marker can help researchers reliably and effectively diagnose pancreatic cancer in patients' bodies. Different fluorescence coding magnetic beads can be achieved by adjusting types and contents of fluorescent dyes in microspheres. These dyes have a same excitation wavelength, but different emission wavelengths, so they can be easily distinguished. In addition, by adjusting proportions of different fluorescent dyes, 100 arrays of different fluorescence coding bead matrices can be formed, and nearly 100 different indexes can be detected at the same time, which greatly improves the detection throughput. Fluorescence coding magnetic beads A and B contain two types of fluorescent dyes, excitation wavelengths of the two types of dyes are 635 nm, and emission wavelengths of fluorescence coding magnetic beads A and B are 658 nm and 712 nm respectively. In addition, the two types of fluorescence coding magnetic beads also contain magnetic particles, which can interact with the magnetic field, thus achieving the operation of capturing the fluorescence coding magnetic beads by magnetic force.

A method for detecting THBS2 and CA19-9 biomarkers in blood at least includes the following detection steps:
(1) a step of forming capture antibody coupled with fluorescence coding magnetic beads, in which two types of fluorescence coding magnetic beads A and B are mixed with THBS2 and CA19-9 capture antibody respectively, and incubated for 30 min-1 h, then the fluorescence coding magnetic beads are separated by magnetic capture to achieve purification of fluorescence coding magnetic bead-capture antibody, and finally a dispersion liquid of THBS2 capture antibody coupled with fluorescence coding magnetic bead A and CA19-9 capture antibody coupled with fluorescence coding magnetic bead B is obtained.
(2) a step of forming fluorescence coding magnetic bead-capture antibody-target molecule coupling, in which the types of magnetic bead antibodies, fluorescence coding magnetic bead A-THBS2 and fluorescence coding magnetic bead B-CA19-9 in equal proportion are mixed, then mixed with a target substance to be detected and incubated for 30 min-1 h, and finally fluorescence coding magnetic bead-capture antibody-target molecule conjugates are obtained through magnetic capture separation and purification.
(3) a step of forming fluorescence coding magnetic bead-capture antibody-target molecule-enzyme labeled detection antibody coupling, in which the dispersion liquid of fluorescence coding magnetic bead-capture antibody-target molecule conjugate is mixed with enzyme label detection antibody and incubated for 30 min-1h, and finally fluorescence coding magnetic bead-capture antibody-target molecule-enzyme label detection antibody conjugates are obtained through magnetic purification.
(4) the dispersion liquid of the purified fluorescence coding magnetic bead-capture antibody-target molecule-enzyme label detection antibody conjugate and a fluorescent substrate are simplified and arrayed, and one drop of fluorescence coding magnetic bead-capture antibody-target molecule-enzyme label detection antibody conjugate is mixed with one drop of fluorescent substrate to form a pL-level reaction system of fluorescence coding magnetic bead and fluorescent substrate capturing a single target molecule, and then the pL reaction system is heated to reduce the reaction system from pL to fL level, and then the heating is stopped. This heating process should take much less time than chemical reaction time of the reaction system.

In the exemplary embodiment, the fluorescence coding magnetic beads A and B and the capture antibodies THBS2 and CA19-9 are added by the first sample addition sub-module 50-1 and the control module 30 into the first mix incubation zone 1011 through the sample addition port for mixing and incubation to form a magnetic bead antibody sample liquid. During the mixing and incubation process in the first mix incubation zone 1011, the first temperature control sub-module 20-1 and the first magnetic control sub-module 40-1 provide required temperature and magnetic force for this process. The dispersion liquid containing magnetic bead antibody flows into purification channels under drive of the control module 30, and the fluorescence coding magnetic beads are separated by magnetic capture of the fourth magnetic control sub-modules 40-4, so as to achieve the purification of magnetic bead antibody (THBS2 capture antibody coupled with fluorescence coding magnetic bead A and CA19-9 capture antibody coupled with fluorescence coding magnetic bead B).

In an exemplary embodiment, the purified dispersion liquid of the magnetic bead antibody enters the second mix incubation zone 1012, and a target substance to be detected is added by the second sample addition sub-module 50-2 and the control module 30 into the second mix incubation zone 1012 at the same time; a sample liquid of the fluorescence coding magnetic bead-capture antibody-target molecule conjugate is formed after mixing and incubation; the second temperature control sub-module 20-2 and the second magnetic control sub-module 40-2 provide required temperature and magnetic force for the mixing and incubation process; then a sample liquid of the fluorescence coding magnetic bead-capture antibody-target molecule conjugate flows into the purification channels, and a dispersion liquid of the fluorescence coding magnetic bead-capture antibody-target molecule conjugate is obtained by magnetic capture of the fourth magnetic control sub-modules 40-4.

In an exemplary embodiment, the purified dispersion liquid of the fluorescence coding magnetic bead-capture antibody-target molecule conjugate enters the third mix incubation zone 1013, and an enzyme label detection antibody is added by the third sample addition sub-module 50-3 and the control module 30 into the third mix incubation zone 1013 at the same time; a sample liquid of fluorescence coding magnetic bead-capture antibody-target molecule-enzyme label detection antibody conjugate is formed after mixing and incubation; the third temperature control sub-module 20-3 and the third magnetic control sub-module 40-3 provide required temperature and magnetic force for the mixing and incubation process, and then a sample liquid of the fluorescence coding magnetic bead-capture antibody-target molecule conjugate flows into the purification channels; and a dispersion liquid of the fluorescence coding magnetic bead-capture antibody-target molecule-enzyme label detection antibody conjugate is obtained by magnetic capture of the fourth magnetic control sub-modules 40-4.

In an exemplary embodiment, when the digital microfluidic device is employed to carry out the above detection, there may be one or more sample addition ports in each functional zone, and the sample addition ports can be disposed in sequence or separately to add samples. The fourth magnetic control sub-modules 40-4 can be separately disposed corresponding to the purification channels connecting the respective functional zones.

In an exemplary embodiment, the purified dispersion liquid of the fluorescence coding magnetic bead-capture antibody-target molecule-enzyme label detection antibody conjugate enters the composite droplet formation zone 1014, and a fluorescent substrate is added by the fourth sample addition sub-module 50-4 and the control module 30 into the composite droplet formation zone 1014 at the same time; simplification and arraying are perform in the composite droplet formation zone 1014; and one drop of the fluorescence coding magnetic bead-capture antibody-target molecule-enzyme label detection antibody conjugate is mixed with one drop of the fluorescent substrate to form a pL-level composite droplet of the fluorescence coding magnetic bead and fluorescent substrate capturing a single target molecule. A composite droplet formed by the fluorescence coding magnetic bead-capture antibody-target molecule-enzyme label detection antibody conjugate and the fluorescent substrate in the treatment zone is heated by the fourth temperature control sub-module 20-4 controlled by the control module 30, and the composite droplet is processed into a target droplet with a droplet diameter less than or equal to 10 μm.

In an exemplary embodiment, after the target droplet is formed, the fluorescence coding magnetic beads A and B are firstly excited using excitation light generated at 635 nm from a fluorescence excitation light source, and filtered by a filter A (658 nm) and a filter B (712 nm) and photographed respectively. Distribution of the fluorescence coding magnetic beads A and B in the digital microfluidic chip can be distinguished by fluorescence images A and B. Then, a fluorescent excitation light source is used to generate excitation light of 532 nm to excite a fluorescent substrate to emit fluorescence, and a filter C (578 nm) is used to collect images to obtain a fluorescence image C. The fluorescence image C reflects the distribution of fluorescence coding magnetic beads capturing target molecules (THBS2, CA19-9) in the digital microfluidic chip. FIG. 14 is a schematic diagram of two-factor joint detection fluorescence images according to an exemplary embodiment of the present disclosure. As shown in FIG. 14, three fluorescence images A, B and C are obtained.

As shown in FIG. 12, three fluorescence images A, B and C are obtained.

In an exemplary embodiment, distribution and quantities of fluorescence coding magnetic beads A and B can be counted from fluorescence images A and B respectively, and distribution and quantities of effective fluorescence coding magnetic beads A-THBS2 and B-CA19-9 can be counted by superimposing A and C, B and C, respectively. Finally, effective statistical values of fluorescence coding magnetic beads A-THBS2 and B-CA19-9 are brought into a standard curve, and low abundance joint detection of THBS2 and CA19-9 molecules in a sample to be detected can be achieved.

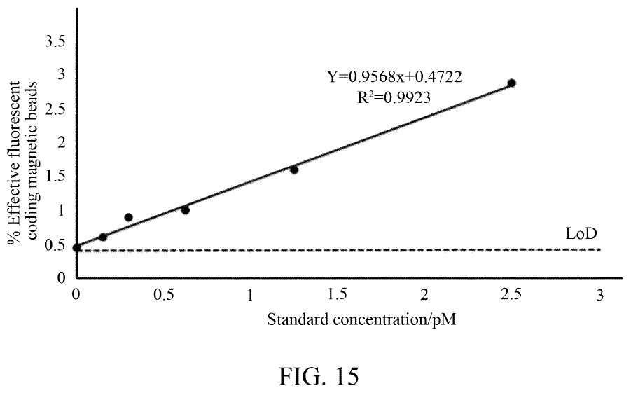

In an exemplary embodiment, the standard curve is obtained by system calibration; THBS2 capture antibody A-THBS2 coupled with fluorescence coding magnetic bead A, CA19-9 capture antibody B-CA19-9 coupled with fluorescence coding magnetic bead B are mixed in equal proportions, then standard THBS2 and CA19-9 are doped into 25% bovine serum solution, diluted into standard samples with concentrations of 0, 0.15, 0.3, 0.625, 1.25 and 2.5 pM, and the standard samples of each concentration are mixed with two types of fluorescence coding magnetic bead capture antibody mixtures, THBS2 with CA19-9 enzyme label detection antibody and fluorescent substrate respectively in step by step; and finally fluorescence coding magnetic bead-capture antibody-target molecule of standard sample-enzyme label detection antibody conjugate is obtained through steps of incubation, purification and dispersion. A proportion of droplets of effective fluorescence coding magnetic beads to total fluorescence coding magnetic beads at different concentrations is respectively detected by a fluorescence imaging module. FIG. 15 is a standard curve of effective fluorescence coding magnetic bead VS standard concentration according to an exemplary embodiment of the present disclosure. As shown in FIG. 15, in an exemplary embodiment, relationship between the proportion of the droplets of effective fluorescence coding magnetic beads to total fluorescence coding magnetic beads and a molecular concentration of a substance to be detected in a large system is nearly linear when the concentration of the substance to be detected is close to pM. Here, a detection limit LoD of the system is determined by testing the sample with concentration of 0 n times (n≥10), an average value of the measured effective fluorescence coding magnetic bead percentage plus 3 times standard deviation is brought into the standard fitting curve in FIG. 15, and the obtained extrapolated solubility of the substance to be detected is the detection limit of this method.

An exemplary embodiment of the present disclosure further provides a detection method of a digital microfluidic device employing the aforementioned digital microfluidic device, including:
forming a composite droplet in the reaction zone of the digital microfluidic chip; and
in the treatment zone of the digital microfluidic chip, controlling the temperature of the temperature control module and the operation mode of the digital microfluidic chip by the control module to process the composite droplet into a target droplet with the droplet diameter less than or equal to 10 μm.

In an exemplary embodiment, the temperature of the temperature control module and the operation mode of the digital microfluidic chip being controlled by the control module to process the composite droplets into target droplets with a droplet diameter less than or equal to 10 μm, includes:
controlling the temperature T of the temperature control module by the control module to be less than or equal to 50° C., and controlling the drive electrodes of the digital microfluidic chip by the control module to alternate between turned-on and turned-off to make the droplet vary between hydrophilic/hydrophobic states during heating, to process the composite droplet into the target droplet with the droplet diameter less than or equal to 10 μm; and
a frequency F at which the drive electrodes alternate between turned-on and turned-off is less than or equal to 50 Hz, and treatment time t for processing the composite droplet is less than 1 min.

In an exemplary embodiment, forming the composite droplet in the reaction zone of the digital microfluidic chip includes:
adding the fluorescence coding magnetic bead and the capture antibody by the sample addition module and the control module into the first mix incubation zone for mixing and incubation to form the magnetic bead antibody sample liquid, which flows into the purification channel, and the fluorescence coding magnetic bead is separated by magnetic capture of a magnetic control module to achieve purification of the magnetic bead antibody;
purified magnetic bead antibody dispersion liquid entering the second mix incubation zone through a purification channel, at the same time adding a target substance to be detected by the sample addition module and the control module into the second mix incubation zone to form fluorescence coding magnetic bead-capture antibody-target molecule conjugate sample liquid after mixing and incubation, and the fluorescence coding magnetic bead-capture antibody-target molecule conjugate sample liquid flowing into the purification channel to be separated and purified through the magnetic capture of the magnetic control module;
the purified fluorescence coding magnetic bead-capture antibody-target molecule conjugate entering the third mix incubation zone through the purification channel, at the same time adding the enzyme label detection antibody by the sample addition module and the control module into the third mix incubation zone to form the fluorescence coding magnetic bead-capture antibody-target molecule-enzyme label detection antibody conjugate sample liquid after mixing and incubation, and the fluorescence coding magnetic bead-capture antibody-target molecule-enzyme label detection antibody conjugate sample liquid flowing into the purification channel to be separated and purified through magnetic capture of the magnetic control module; and
mixing the purified fluorescence coding magnetic bead-capture antibody-target molecule-enzyme label detection antibody conjugate with a fluorescent substrate to obtain a complex droplet.

In an exemplary embodiment, the detection method further includes:
performing fluorescence detection on the target droplet by the signal detection module and transmitting detection information to the processing modules to obtain concentration information.

An exemplary embodiment of the present disclosure further provides a method for screening a single cell employing the aforementioned digital microfluidic device, including:
forming a droplet containing the single cell in the reaction zone of the digital microfluidic chip, and at least part of the droplet contains the single cell;
driving the droplet by the drive electrodes to move to the treatment zone for processing;
controlling the temperature control module by the control module to provide a set temperature to the treatment zone to heat the droplet, and controlling the drive electrodes located in the treatment zone by the control module to alternate between turned-on and turned-off to make a solid-liquid contact surface at a position where the droplet is located vary between hydrophilic/hydrophobic states; reducing a diameter of the droplet by heating and change of hydrophilic/hydrophobic states; the droplet with reduced diameter including a target droplet containing at most one of the single cell;
screening out a target droplet containing the single cell using optical difference of the target droplet.

In an exemplary embodiment, controlling the temperature control module by the control module to provide the set temperature to the treatment zone to hat the droplet, and controlling the drive electrodes located in the treatment zone by the control module to alternate between turned-on and turned-off includes:
controlling the drive electrodes located in the treatment zone by the control module to alternate between turned-on and turned-off to make the droplet vary between hydrophilic/hydrophobic states during heating, and processing the composite droplet into a target droplet with a diameter of 20 μm to 50 μm; and
a frequency F at which the drive electrodes alternate between turned-on and turned-off is less than or equal to 50 Hz, and treatment time t for processing the droplet is less than 1 min.

In an exemplary embodiment, the method for screening single cell further includes: controlling the temperature T of the temperature control module by the control module to be less than or equal to 50° C.

In an exemplary embodiment, the method for screening the single cell further includes: after screening out the target droplet containing the single cell,
adding a target antigen to the target droplet to make the single cell in the target antibody secrete a target antibody; and
screening out a target droplet containing the target antibody based on an optical difference between the target droplet containing the target antibody and a target droplet without containing the target antibody.

An exemplary embodiment of the disclosure further provides a library creation and detection method employing the aforementioned digital microfluidic device, including:
forming a composite droplet containing a library in the reaction zone of the digital microfluidic chip;
driving the composite droplet by the drive electrodes to move to the treatment zone for processing;
controlling the drive electrodes located in the treatment zone by the control module to alternate between turned-on and turned-off to make the solid-liquid contact surface at the position where the composite droplet is located vary between hydrophilic/hydrophobic states; reducing a diameter of the composite droplet by heating and change of hydrophilic/hydrophobic states;
detecting nucleic acid content and quality of the target droplet by using optical difference of the target droplet, thereby obtaining the nucleic acid content and the quality of the composite droplet (for example, a concentration and purity of the nucleic acid in the composite droplet, etc.).

In an exemplary embodiment, controlling the drive electrodes located in the treatment zone by the control module to alternate between turned-on and turned-off includes:
controlling the drive electrodes located in the treatment zone by the control module to alternate between turned-on and turned-off to make the droplet vary between hydrophilic/hydrophobic states during heating, and processing the composite droplet into a target droplet with a diameter of less than or equal to 100 μm;
a frequency F at which the drive electrodes alternate between turned-on and turned-off is less than or equal to 50 Hz, and treatment time t for processing the droplet is less than 1 min.

In an exemplary embodiment, the library creation and detection method further include: controlling the temperature control module by the control module to provide a set temperature to the treatment zone to heat the composite droplet.

In an exemplary embodiment, controlling the temperature control module by the control module to provide the set temperature to the treatment zone includes: controlling the temperature T of the temperature control module by the control module to be less than or equal to 50° C.

Although the implementation modes disclosed in the present disclosure are as above, the described contents are only implementation modes used for convenience of understanding the present disclosure and are not intended to limit the present disclosure. Any of those skilled in the art of the present disclosure can make any modifications and variations in the implementation manner and details without departing from the spirit and scope of the present disclosure. However, the protection scope of the present disclosure should be subject to the scope defined by the appended claims.

1. A drive method of a digital microfluidic device, an array element of the digital microfluidic device having drive electrodes and a reference electrode, the drive method comprising:
applying a first reference voltage to the reference electrode; and
addressing the drive electrode according to a set of data, comprising:
(i) when a first scan voltage is applied, a first data voltage is written to a corresponding drive electrode in an array to define a voltage difference with a magnitude equal to or greater than an actuation voltage between two ends of the array element, and the array element is in an actuated state;
(ii) when a second scan voltage is applied, the first data voltage is electrically isolated from the corresponding drive electrode in the array to define a voltage difference with a magnitude less than the actuation voltage between the two ends of the array element, and the array element is in a non-actuated state;
(iii) alternately writing the first scan voltage and the second scan voltage to the array element to make the array element be alternately in the actuated state and the non-actuated state;
wherein in the actuated state, the array element is configured to actuate a droplet in the array element, and in the non-actuated state, the array element is configured not to actuate the droplet in the array element;
the droplet in the array element is processed into a target droplet having a diameter smaller than a diameter of the droplet by alternately performing actuation and non-actuation processing.

2. The drive method of the digital microfluidic device according to claim 1, wherein,
the first scan voltage is a valid level; and
the second scan voltage is an invalid level.

3. The drive method of the digital microfluidic device according to claim 2, further comprising: forming the droplet in the digital microfluidic device;
the array element is made to be alternately in the actuated state and the non-actuated state to make a solid-liquid contact surface at a position where the droplet is located vary between hydrophilic/hydrophobic states.

4. The drive method of the digital microfluidic device according to claim 3, further comprising: heating the droplet by using a temperature control module to reduce the diameter of the droplet to obtain the target droplet.

5. The drive method of the digital microfluidic device according to claim 1, wherein the diameter of the target droplet is less than or equal to 10 μm; or
the diameter of the target droplet is 20 μm to 50 μm; or
the diameter of the target droplet is less than or equal to 100 μm; or
a frequency F at which the drive electrodes alternate between turned-on and turned-off is less than or equal to 50 Hz.

6-8. (canceled)

9. The drive method of the digital microfluidic device according to claim 4, wherein a temperature T at which the droplet is heated is less than or equal to 50° C.

10. The drive method of the digital microfluidic device according to claim 1, wherein treatment time t for processing the droplet is less than 1 min.

11. A digital microfluidic device comprising a digital microfluidic chip, the digital microfluidic chip comprising at least a drive electrode and a reference electrode;
the reference electrode is configured to write a first reference voltage;
the drive electrode is configured to alternately write a first scan voltage and a second scan voltage so as to be alternately in an actuated state and a non-actuated state, and in the actuated state, the drive electrode is configured to actuate a composite droplet in the digital microfluidic chip, and in the non-actuated state, the drive electrode is configured not to actuate the composite droplet in the digital microfluidic chip; and
the composite droplet is processed into a target droplet having a diameter smaller than a diameter of the composite droplet by alternately performing actuation and non-actuation processing.

12. The digital microfluidic device according to claim 11, further comprising: a temperature control module and a control module, the digital microfluidic chip further comprises a reaction zone and a treatment zone, the reaction zone is configured to form the composite droplet, and the treatment zone is configured to process the composite droplet; the temperature control module is configured to provide a set temperature for the treatment zone, the control module is connected to the digital microfluidic chip and the temperature control module, and the control module is configured to control a temperature of the temperature control module and control an operation mode of the digital microfluidic chip to process the composite droplet in the treatment zone into the target droplet.

13. The digital microfluidic device according to claim 12, wherein a cell thickness of the drive electrode and the digital microfluidic chip satisfies following formula:
$$\frac{L}{H} = - \sqrt{2} tan θ$$
wherein θ represents an initial contact angle between the droplet and a hydrophobic surface, H represents a cell thickness of the digital microfluidics chip, and L represents a size of the drive electrode.

14. The digital microfluidic device according to claim 13, wherein the diameter of the target droplet is less than or equal to 10 μm.

15. The digital microfluidic device according to claim 13, wherein the diameter of the target droplet is 20 μm and 50 μm.

16. The digital microfluidic device according to claim 13, wherein the diameter of the target droplet is less than or equal to 100 mum; or
the operation mode of the digital microfluidic chip is as follows: controlling the drive electrode by the control module to alternate between turned-on and turned-off to make a solid-liquid contact surface at a position where the droplet is located vary between hydrophilic/hydrophobic states during heating.

17. The digital microfluidics chip according to claim 14, wherein the cell thickness H of the digital microfluidics chip is less than or equal to 10 μm and the size L of the drive electrode is less than or equal to 12.25 μm.

18. The digital microfluidics chip according to claim 15, wherein the cell thickness H of the digital microfluidics chip is 10 μm to 30 μm and the size L of the drive electrode is 12 μm to 50 μm.

19. The digital microfluidic device according to claim 16, wherein the cell thickness H of the digital microfluidic chip is 30 μm to 200 μm and the size L of the drive electrode is 50 μm to 2 mm.

20. (canceled)

21. A detection method using the digital microfluidic device according to claim 14, comprising:
forming the composite droplet in the reaction zone of the digital microfluidic chip; and
controlling the drive electrode of the digital microfluidic chip by the control module to alternate between turned-on and turned-off to make a solid-liquid contact surface at a position where the droplet is located vary between hydrophilic/hydrophobic states during heating, to process the composite droplet into the target droplet with the diameter less than or equal to 10 μm; and
a frequency F at which the drive electrode alternates between turned-on and turned-off is less than or equal to 50 Hz, and treatment time t for processing the composite droplet is less than 1 min.

22. A method for screening single cell using the digital microfluidic device according to claim 15, comprising:
forming a droplet containing the single cell in the reaction zone of the digital microfluidic chip, and at least part of the droplet contains the single cell;
driving the droplet by the drive electrode to move to the treatment zone for processing;
controlling the drive electrode located in the treatment zone by the control module to alternate between turned-on and turned-off to make a solid-liquid contact surface at the position where the droplet is located vary between hydrophilic/hydrophobic states during heating, thereby reducing the diameter of the droplet to 20 μm to 50 μm; the droplet with reduced diameter comprising a target droplet containing at most one of the single cell;
screening out a droplet containing the single cell using optical difference of the target droplet;
wherein, a frequency F at which the drive electrode alternates between turned-on and turned-off is less than or equal to 50 Hz, and treatment time t for processing the droplet is less than 1 min.

23. A library creation and detection method using the digital microfluidic device according to claim 16, comprising:
forming a composite droplet containing a library in the reaction zone of the digital microfluidic chip;
driving the composite droplet by the drive electrode to move to the treatment zone for processing;
controlling the drive electrode located in the treatment zone by the control module to alternate between turned-on and turned-off to make a solid-liquid contact surface at a position where the composite droplet is located vary between hydrophilic/hydrophobic states, thereby reducing the diameter of the composite droplet to less than or equal to 100 μm;
detecting nucleic acid content and quality of the target droplet by using optical difference of the target droplet, thereby obtaining the nucleic acid content and the quality of the composite droplet;
wherein a frequency F at which the drive electrode alternates between turned-on and turned-off is less than or equal to 50 Hz, and treatment time t for processing the droplet is less than 1 min.

24. The method according to claim 21, further comprising: controlling the temperature T of the temperature control module by the control module to be less than or equal to 50° C.
